(19) United States
(12) Patent Application Publication
(10) Pub. No.: US 2007/0249867 A1
(43) Pub. Date: Oct. 25, 2007

(54) Method for Synthesis of Aromatic Amine

(76) Inventors: Harue Nakashima, Kanagawa (JP); Sachiko Kawakami, Kanagawa (JP)

Correspondence Address: ERIC ROBINSON, PMB 955, 21010 SOUTHBANK ST., POTOMAC FALLS, VA, 20165, US

(21) Appl. No.: 11/658,411

(22) PCT Filed: Aug. 8, 2005
(86) PCT No.: PCT/JP05/14867

§ 371 Date: Jan. 25, 2007

(30) Foreign Application Priority Data
Aug. 11, 2004 (JP) 2004-234860

(51) Int. Cl. C07C 209/10

(52) U.S. Cl. 564/405

(57) ABSTRACT

One embodiment of the present invention provides a method for synthesis of substituted secondary amine by the reaction of aniline with aryl halide by using a Pd catalyst including (t-Bu)₃P as a ligand.

### TECHNICAL FIELD

The present invention relates to a method for synthesis of an aromatic amine compound. More specifically, the present invention relates to a method for synthesis of N-(4-diphenylamino)phenylaniline.

### BACKGROUND ART

In recent years, a so-called self luminous type display device in which a pixel is formed by a light-emitting element such as a light-emitting diode (LED) is attracted attention. As a light-emitting element used for such the self luminous type display device, an organic light-emitting diode (also referred to as an OLED (Organic Light Emitting Diode), an organic EL element, or electroluminescence (Electro Luminescence: EL)) is attracted attention and used for an organic EL display or the like. Such the self luminous type display device has advantages of high response speed, excellent movie display, and a wide viewing angle in addition to the advantages in the existing liquid crystal display device, and attracts attention as a next generation flat panel display.

The light-emitting element has a layer containing a light-emitting substance which generates luminescence by being applied with an electric field (Electroluminescence), an anode, and a cathode. The luminescence is obtained by recombining holes injected from the anode with electrons injected from the cathode within the layer containing a light-emitting substance. The luminescence generated in the layer containing a light-emitting substance includes light emission (fluorescence) radiated while returning from a singlet excited state to a ground state, and light emission (phosphorescence) radiated while returning from a triplet excited state to a ground state.

There are inorganic light-emitting material and an organic light-emitting material as a light-emitting material used for a light-emitting element. The organic light-emitting material is attracted attention since it requires low driving voltage.

The structure of the foregoing light-emitting element is required to be optimized to achieve driving voltage reduction, long lifetime, color purity improvement, and the like. Generally, the layer containing a light-emitting substance has a laminated structure, typically, hole transporting layer/light-emitting layer/electron transporting layer. Since the laminated structure has extremely high light emission efficiency, almost light-emitting devices being developed at present adopt the laminated structure. Besides, laminated structures such as hole injecting layer/hole transporting layer/light-emitting layer/electron transporting layer and hole injecting layer/hole transporting layer/light-emitting layer/electron transporting layer/electron injecting layer are adopted.

Many substances are known as a material for constituting the foregoing layers. For example, aromatic amine compounds (that is, having the bond of benzene ring and nitrogen) or the like are used for the hole transporting layer. The aromatic amine compounds have an aromatic amine skeleton with a high hole accept property, and so the aromatic amine compounds have a high hole transporting property. Similarly, substance having an aromatic amine skeleton can be considered as having a high hole transporting property. Hence, a substance having an aromatic amine skeleton can be used for a light-emitting substance, a substance for dispersing a light-emitting substance, or the like.

As a method for synthesis of the aromatic amine, a method using amine and aryl halide is generally used. The case that aniline which is primary amine is used as the amine to synthesize substituted secondary amine is considered.

The aniline has two N—H bonds which are reaction sites. Accordingly, in the case that reaction is conducted without introducing a protective group for protecting either of the reaction sites, not only substituted amine but also disubstituted tertiary amine may be synthesized. Therefore, a way of introducing a protective group to either of the reaction sites of aniline has been adopted to synthesize substituted amine (refer to patent document 1).

Patent document: Unexamined patent publication No. 2003-238501

However, when substituted secondary amine is synthesized according to the foregoing method, the reaction requires three steps, that is, a step of introduction of a productive group, a step of reaction of aniline introduced with the protective group and aryl halide, and a step of elimination of the protective group. Therefore, long time, high costs, and much energy are required for the synthesis, and an object is obtained in a low yield.

In recent years, aniline can react with aryl halide to obtain substituted amine in a high yield by using a Pd catalyst without introducing a protective group (refer to non-patent document 1).

Non patent document 1: John F. Hartwig, “Angewandte Chemistry International Edition”, 1998, 37, 2046-2067

As disclosed in the non patent document 1, substituted secondary amine can be obtained in a high yield by the reaction of aniline which is not introduced with a protective group with aryl halide in the case of using second generation Pd catalyst, whereas substituted secondary amine is hardly obtained in the case of using first generation Pd catalyst.

The second generation Pd catalyst is a complex between DPPF (1,1′-bis-(diphenylphosphino)ferrocene) or BINAP (2,2′-bis-(diphenylphosphino)-1,1′-binaphthyl) and Pd. The synthesis of complex requires so much time and energy. Further, the second generation Pd catalyst is expensive.

On the other hand, the first generation Pd catalyst is a Pd catalyst which uses a triphenylphosphine or a tris(o-tolyl)phosphine as a ligand. As mentioned above, a substrate is limited to obtain substituted secondary amine from the aryl halide and aniline efficiently.

### DISCLOSURE OF INVENTION

In view of the foregoing, it is an object of the present invention to provide a synthesis method which can obtain easier substituted secondary amine by the reaction of aniline with aryl halide without introducing a protective group than the conventional method.

One embodiment of the present invention provides a method for synthesis of substituted secondary amine by the reaction of aniline with aryl halide in by using a Pd catalyst including (t-Bu)₃P as a ligand. That is, the substituted secondary amine is synthesized by the reaction of aniline with aryl halide by using a Pd catalyst including (t-Bu)₃P as a ligand.

Another embodiment of the present invention provides a method for synthesis of secondary amine by heating aniline and aryl halide by using a Pd catalyst including (t-Bu)₃P as a ligand.

In the foregoing method, the aryl halide is N,N-diphenyl-N-(4-bromophenyl)amine.

Another embodiment of the present invention provides a method for synthesis of N-(4-diphenylamino)phenylaniline by heating aniline and N,N-diphenyl-N-(4-bromophenyl)amine in the presence of by using a Pd catalyst including (t-Bu)₃P as a ligand.

In the foregoing method, reaction temperature is from 60 to 110° C.

### BRIEF DESCRIPTION OF DRAWINGS

FIG. 1 is a view for showing a ¹H-NMR chart for N-(4-diphenylamino)phenylaniline which is synthesized by a synthesis method according to the present invention;

FIG. 2 is a view for showing a ¹³C-NMR chart for N-(4-diphenylamino)phenylaniline which is synthesized by a synthesis method according to the present invention;

FIG. 3 is a view for showing a result of thermogravimetry for N-(4-diphenylamino)phenylaniline which is synthesized by a synthesis method according to the present invention;

FIG. 4 is a view for showing a ¹H-NMR for N-(4-diphenylamino)phenylacetanilide which is synthesized by the conventional synthesis method; and

FIGS. 5A to 5C are diagrams of an electronic device mounted with a display device to which the present invention is applied.

### BEST MODE FOR CARRYING OUT THE INVENTION

A synthesis method according to the present invention is heating and stirring aniline and aryl halide by using a Pd catalyst including (t-Bu)₃P as a ligand For example, (t-Bu)₃P is coordinated with Pd by mixing of Pd(dba)₂ and (t-Bu)₃P. Alternatively, a Pd complex which is coordinated with a ligand having smaller coordination ability than (t-Bu)₃P may be used. Example of the Pd complex include, but are not limited to, PdCl₂(PhCN)₂, Pd(OAc)₂ and the like. Heating temperature is preferably in the range of from room temperature to 130° C. In the case of heating to 130° C. or more, the Pd(dba)₂ is resolved and cannot be served as a catalyst. The heating temperature is preferably set from 60 to 110° C., since reaction becomes easily controlled and a yield is improved. As used herein, the term “dba” refers to trans, trans-dibenzylideneacetone. As solvent, dioxane or the like can be used. As a base, alkali metal alcoxide or the like such as t-BuONa can be used.

By the synthesis method according to the present invention, substituted secondary amine can be synthesized without introducing a protective group to aniline. That is, secondary amine can be synthesized by one step of reaction without passing through three steps, that is, a step of introduction of a productive group, a step of reaction of aniline introduced with the protective group and aryl halide, and a step of elimination of the protective group.

Substituted secondary amine can be synthesized without using the second generation Pd catalyst which is acquired requiring energy, time, and costs.

As mentioned above, the secondary amine explained in the present invention can be used for a display device including a light-emitting element (an Organic Light-Emitting Diode, an organic EL element, or an Electro Luminescence element, etc.). It may be an active-type display device to control driving the light-emitting element with a transistor, alternatively, a passive-type display device to control driving the light-emitting element without especially providing an element for driving such as transistors.

### EXAMPLE 1

Hereinafter, a method for synthesis of N-(4-diphenylamino)phenylaniline by the synthesis method according to the present invention is explained.

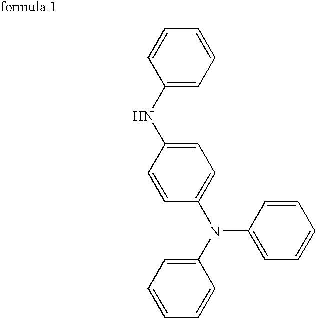

A method for synthesis of N-(4-diphenylamino)phenylaniline represented by the structural formula 1 is explained.

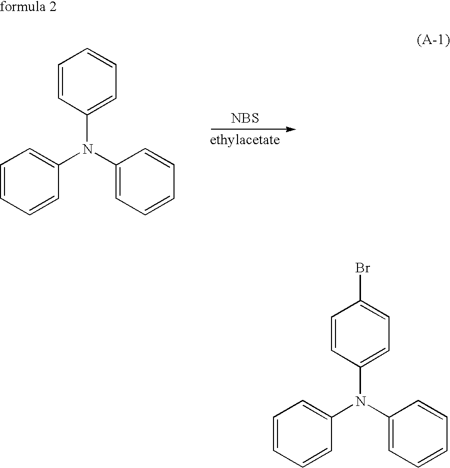

According to the foregoing reaction scheme (A-1), N,N-diphenyl-N-(4-bromophenyl)amine was firstly synthesized. Triphenylamine (25.19 g, 0.102 mol), N-bromosuccinimide (18.05 g, 0.102 mol), and ethyl acetate (400 ml) were added to a 1000 ml Erlenmyer flask and the solution was stirred at room temperature in the air all night. After the reaction, the reacted solution washed with saturated sodium carbonate twice to extract a water layer with ethyl acetate twice. The water layer and an organic layer were washed with saturated salt solution. After drying the organic layer with magnesium sulfate, the organic layer was filtered naturally and condensed to give colorless solid. The colorless solid was recrystallized with ethyl acetate-hexane and 22.01 g of colorless powder solid was obtained in a yield of 66%. ¹H-NMR of the white powder solid was carried out to confirm that the white powder solid was N,N-diphenyl-N-(4-bromophenyl)amine and the result was as follows:

¹H NMR (300 MHz, CDCl₃) δ ppm: 7.32 (d, 2H, J=8.7 Hz), 7.29-7.23 (m, 5H), 7.08-7.00 (m, 6H), 6.94 (d, 2H, J=8.7 Hz)

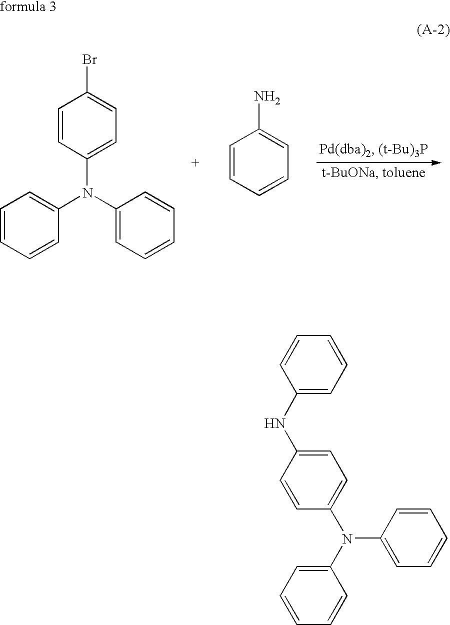

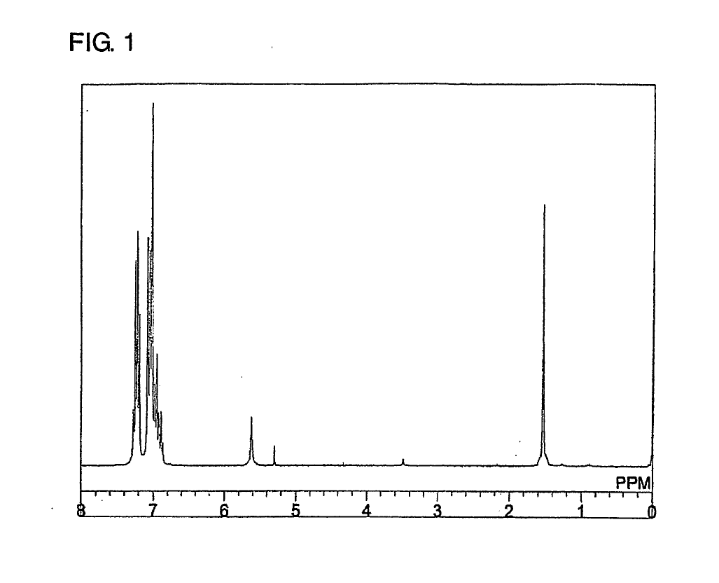

According to the foregoing reaction scheme (A-2), coupling the N,N-diphenyl-N-(4-bromophenyl)amine to aniline was carried out to synthesize N-(4-diphenylamino)phenylaniline which is an object of the present invention. Dehydrated toluene solution (5 ml) of N,N-diphenyl-N-(4-bromophenyl)amine (559 mg, 6 mmol), Pd (dba)₂ (345 mg, 0.6 mmol), and t-BuONa (577 mg, 6 mmol) was deaerated, and aniline (559 mg, 6 mmol) and P(t-Bu)₃ (0.37 ml, 1.8 mmol) were added to the solution, then the mixture was heated to stir at 80° C. under nitrogen atmosphere for 5 hours. Thin-layer chromatography confirmed that a raw material, 4-bromotriphenylamine, was lost. The reaction was terminated by adding saturated salt solution and a water layer was extracted with approximately 100 ml of ethyl acetate. An organic layer was dried with magnesium sulfate and filtered. After condensing the filtrate, the filtrate was passed through silica gel column (ethyl acetate: hexane=1:20) to be purified. Then, an object as viscous fluid was obtained. The viscous fluid was added with hexane and exposed to ultrasound to separate cream-colored powder therefrom. The mixture was condensed to obtain the above mentioned compound N-(4-diphenylamino)phenylaniline in a yield of 42% based on the N,N-diphenyl-N-(4-bromophenyl)amine. FIG. 1 shows a ¹H-NMR chart of the obtained N-(4-diphenylamino)phenylaniline and the result was as follows:

¹H NMR (300 MHz, CDCl₃) δ ppm: 7.35-6.83 (m, 19H), 5.60 (s, 1H)

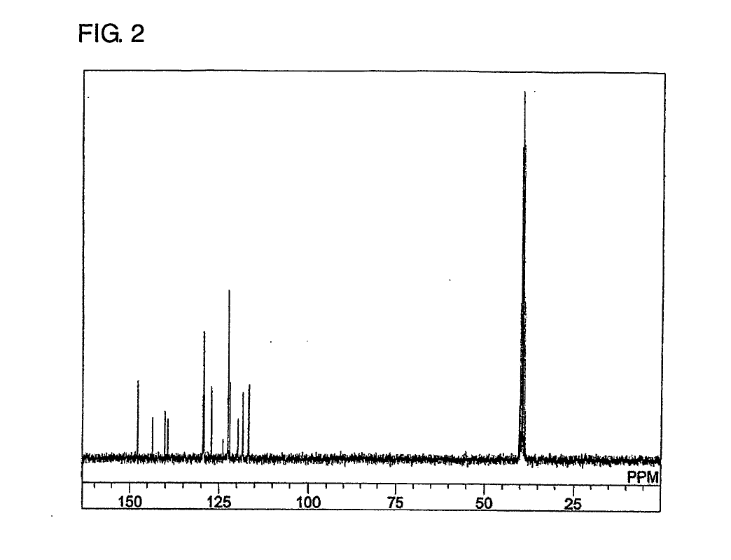

FIG. 2 shows ¹³C-NMR chart of the N-(4-diphenylamino)phenylaniline and the result was as follows:

¹³C NMR (75.5 MHz, DMSO-d6) δ ppm: 147.8, 143.7, 140.2, 139.4, 129.4, 129.3, 127.1, 122.4, 122.0, 119.8, 118.4, 116.8

The melting point of the obtained N-(4-diphenylamino)phenylaniline was measured by melting-point apparatus (manufactured by AS ONE CORPORATION, ATM-01) and the result was that the melting point was 105 to 106° C.

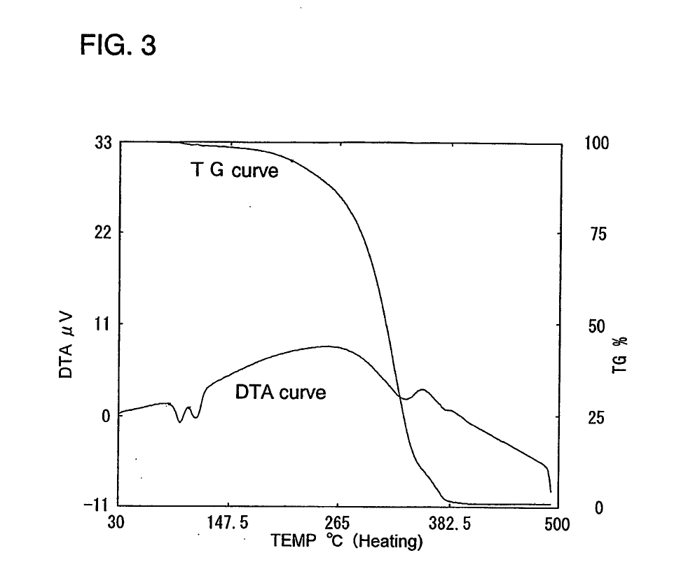

FIG. 3 shows the result of thermogravimetry-differential thermal analysis (TG-DTA) of the N-(4-diphenylamino)phenylaniline. In FIG. 3, a left hand vertical axis represents calorie, whereas a right hand vertical axis represents weight (%; weight represented on the basis that the weight at onset of measurement as 100%). Further, a horizontal axis represents temperature (° C.). Thermo-Gravimetric/Differential Thermal Analyzer (SII Nano Technology Inc., TG/DTA-320) was used for the measurement, which measured thermophysical properties under nitrogen atmosphere at heating rate of 10° C./min. As a result, from the relationship between the weight and the temperature (thermogravimetry), the temperature at which the weight is 95% or less against the weight at the onset of measurement at normal pressure was 214° C.

As mentioned above, the secondary amine explained in the present invention can be used for a display device including a light-emitting element (an Organic Light-Emitting Diode, an organic EL element, or an Electro Luminescence element, etc.). It may be an active-type display device to control driving the light-emitting element with a transistor, alternatively, a passive-type display device to control driving the light-emitting element without especially providing an element for driving such as transistors.

### EXAMPLE 2

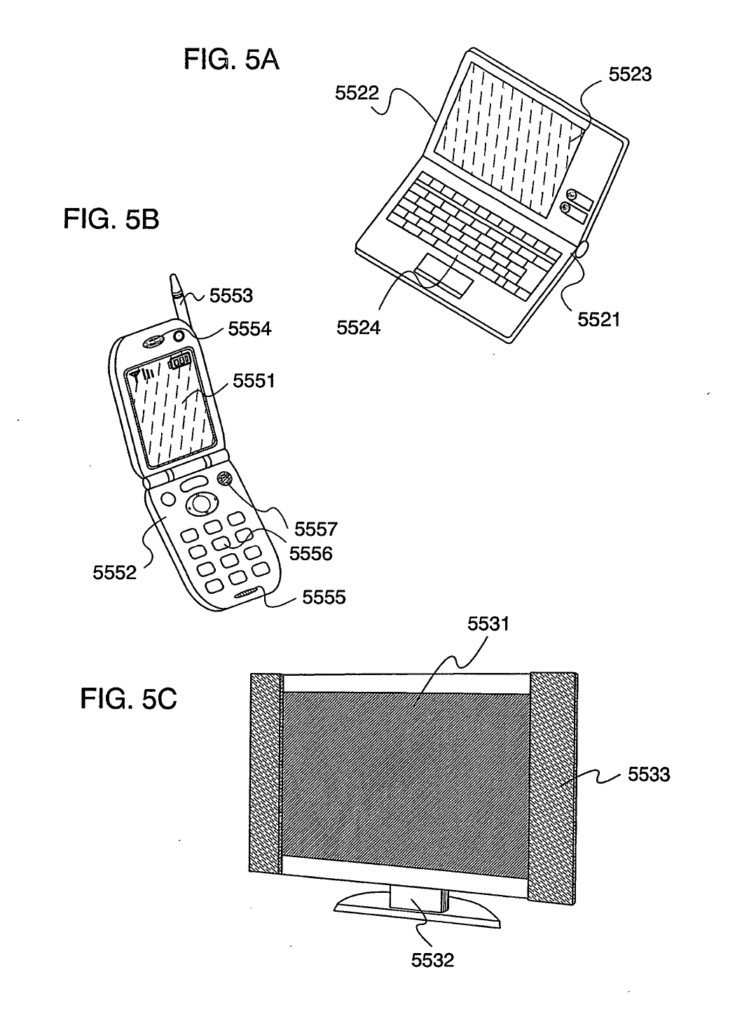

FIG. 5 shows an example of electrical device that mount with a display device that has a light-emitting element using the secondary amine of present invention.

FIGS. 5A to 5C show examples of an electronic device mounted with a display device to which the present invention is applied.

FIG. 5A shows a laptop personal computer manufactured according to the present invention, which includes a main body 5521, a frame body 5522, a display portion 5523, and a keyboard 5524. The personal computer can be completed by incorporating a display device that has a light-emitting element using the secondary amine according to the present invention into the display portion 5523.

FIG. 5B shows a cellular phone manufactured according to the present invention, which includes a main body 5552, a display portion 5551, a voice output portion 5554, a voice input portion 5555, operation keys 5556 and 5557, and an antenna 5553. The cellular phone can be completed by incorporating a display device that has a light-emitting element using the secondary amine according to the present invention into the display portion 5551.

FIG. 5C shows a television manufactured according to the present invention, which includes a display portion 5531, a frame body 5532, and a speaker 5533. The television can be completed by incorporating a display device that has a light-emitting element using the secondary amine according to the present invention into the display portion 5531.

As described above, a display device according to the present invention is suitable for use as display portions of various electronic devices.

Further, in addition to the electronic device described above, a display device that has a light-emitting element using the secondary amine according to the present invention may be mounted in devices such as a navigation system and a lighting apparatus.

### COMPARATIVE EXAMPLE

As a comparative example, a synthesis example according to the conventional method is explained. A reaction scheme (A-3) is as follows:

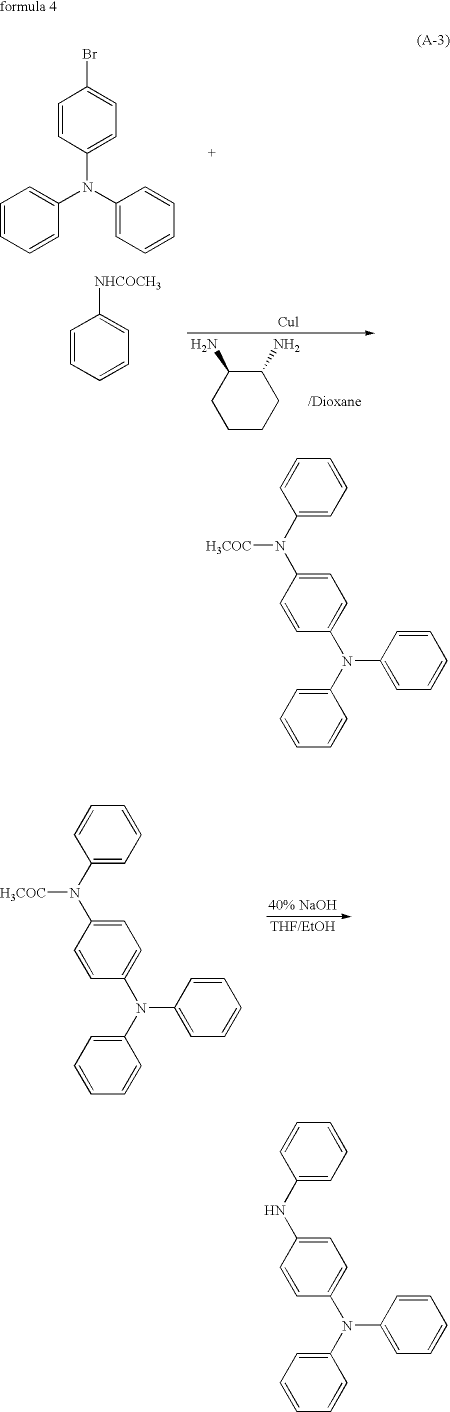

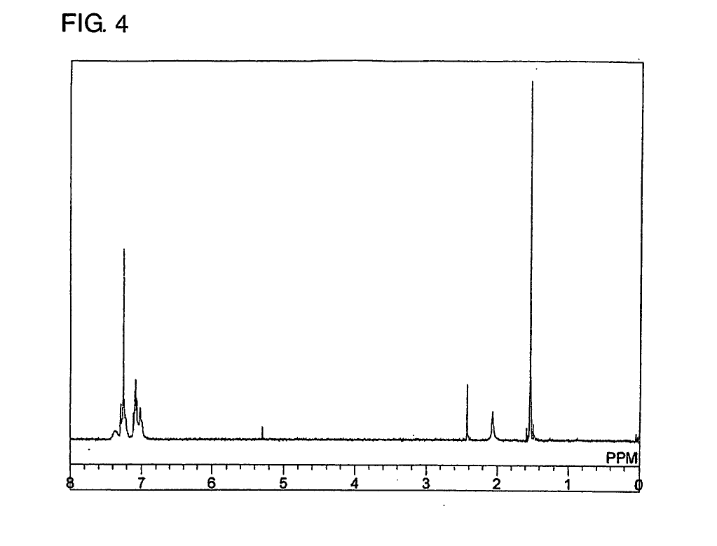

Under nitrogen gas stream, 1.3 ml of trans-1,2-cyclohexanediamine was added to 150 ml of dioxane suspension of acetanilide (7.21 g, 0.053 mol), N,N-diphenyl-N-(4-bromophenyl)amine (17.32 g, 0.053 mol), copper iodide (2.05 g, 0.011 mol), and tripotassium phosphate (22.00 g, 0.103 mol), and the solution was heated to be refluxed for 40 hours. After the reaction, the solution was cooled to room temperature and a solid within the system was removed by suction filtration. The solution washed with saturated sodium carbonate solution twice and a water layer was extracted with chloroform twice. The water layer and an organic layer were washed with saturated salt solution. After drying the organic layer with magnesium sulfate, the organic layer was filtered naturally and condensed. Colorless solid was obtained and the obtained colorless solid was passed through silica gel column (ethyl acetate: hexane=1:1) to be purified. Then, 12.00 g of white powder solid was obtained in a yield of 59%. ¹H-NMR of the white powder solid was carried out to confirm that the solid was N-(4-diphenylamino)phenylacetanilide. FIG. 4 shows the ¹H-NMR chart of the compound. Further, the ¹H-NMR data was as follows:

¹H NMR (300 MHz, CDCl₃) δ ppm: 7.36-7.23 (m, 9H), 7.12-7.03 (m, 10H), 2.07 (s, 3H)

Subsequently, the synthesized N-(4-diphenylamino)phenylacetanilide (20.00 g, 0.053 mol), 100 g of 40% sodium hydroxide solution, 50 ml of tetrahydrofuran, and 50 ml of ethanol are heated to be refluxed for 2 hours in the air. After the reaction, the solution was cooled to room temperature, and a water layer and an organic layer were isolated. The organic layer washed with water twice. On the other hand, the water layer was extracted with chloroform twice. The chloroform layer and the organic layer were washed with saturated salt solution. After drying the chloroform layer and the organic layer with magnesium sulfate, the layers were filtered naturally and condensed to give a colorless solid. The obtained colorless solid was recrystallized with ethyl acetate-hexane and 14.69 g of colorless powder solid was obtained in a yield of 83%. ¹H-NMR of the white powder solid was carried out to confirm that the white powder solid was N-(4-diphenylamino)phenylaniline.

According to the conventional synthesis method explained in the comparative example, N,N-diphenyl-N-(4-bromophenyl)amine having a bromo group and acetanilide introduced with a protective group were reacted with each other under the catalytic influence of Cu, accordingly, the reaction could not terminate unless being refluxed at high temperature for 40 hours. On the other hand, N,N-diphenyl-N-(4-bromophenyl)amine and aniline which is not protected by a protective group were reacted with each other in the presence of Pd(dba)₂ and (t-Bu)₃P which are the first generation Pd catalysts by the synthesis method according to the present invention, accordingly, the reaction was proceeded by heating and stirring at 80° C. for 5 hours. That is, the synthesis method according to the present invention can precede reaction at lower temperature and a shorter time than those in the conventional method, and so synthesis time can be drastically shorted.

Since the synthesis can be conducted using Pd(dba)₂ which is the first generation Pd catalyst, time, energy, and costs for the synthesis can be saved compared to the case of using the second generation catalyst.

The present application is based on Japanese Priority Application No. 2004-234860 filed on Aug. 11, 2004 with the Japan Patent Office, the entire contents of which are hereby incorporated by references.

1. A method for synthesis of aromatic amine comprising:
reacting aniline with aryl halide by using a Pd catalyst including (t-Bu)₃P as a ligand to obtain secondary amine.

2. A method for synthesis of aromatic amine comprising:
heating aniline and aryl halide by using a Pd catalyst including (t-Bu)₃P as a ligand to obtain secondary amine.

3. A method for synthesis of aromatic amine comprising:
reacting aniline with aryl halide in presence of Pd(dba)₂ and (t-Bu)₃P to obtain secondary amine.

4. The method for synthesis of aromatic amine according to claim 1 to 3, wherein the aryl halide is N,N-diphenyl-N-(4-bromophenyl)amine.

5. A method for synthesis of aromatic amine comprising:
heating aniline and N,N-diphenyl-N-(4-bromophenyl)amine by using a Pd catalyst including (t-Bu)₃P as a ligand to obtain N-(4-diphenylamino)phenylaniline.

6. The method for synthesis of aromatic amine according to any one of claims 2 and 5, wherein reaction temperature is from 60 to 110° C.
